(19) United States
(12) Patent Application Publication
(10) Pub. No.: US 2015/0258118 A1
(43) Pub. Date: Sep. 17, 2015

(54) CO-MICRONISATION PRODUCT COMPRISING A SELECTIVE PROGESTERONE-RECEPTOR MODULATOR

(71) Applicant: LABORATOIRE HRA-PHARMA, Paris (FR)

(72) Inventors: Florian Battung, Paris (FR); Pierre-Yves Juvin, Nantes (FR); Jérôme Hecq, Camblanes et Meynac (FR); Aude Colin, Bordeaux (FR)

(21) Appl. No.: 14/441,054

(22) PCT Filed: Nov. 7, 2013
(86) PCT No.: PCT/FR2013/052671

§ 371 Date: May 6, 2015

(30) Foreign Application Priority Data
Nov. 8, 2012 (FR) 1260605

(51) Int. Cl. A61K 31/57; A61K 9/48; A61K 9/14

(52) U.S. Cl. CPC A61K 31/57; A61K 9/146; A61K 9/4825

(57) ABSTRACT

The subject of the present invention is a co-micronization product comprising: an active ingredient selected from the group consisting of selective progesterone receptor modulators, metabolites thereof and mixtures thereof, and an N-vinyl-2-pyrrolidone-based polymeric excipient. The invention also relates to a pharmaceutical composition comprising said co-micronization product and to the therapeutic uses thereof.

### FIELD OF THE INVENTION

The present invention relates to a novel galenic form of a selective progesterone receptor modulator (SPRM), more specifically to a co-micronization product, and to pharmaceutical compositions containing said galenic form.

### TECHNICAL BACKGROUND OF THE INVENTION

Ulipristal acetate (abbreviated as UPA) corresponds to 17α-acetoxy-11β-(4-N,N-dimethylaminophenyl)-19-norpregna-4,9-diene-3,20-dione (IUPAC nomenclature) and has the following chemical formula:

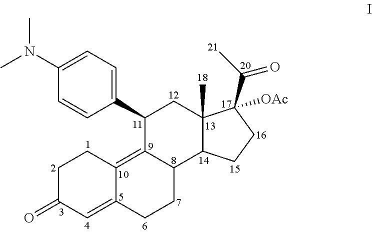 [SMILES: CC(=O)O[C@]1(C(C)=O)CCC2C3CCC4=CC(=O)CCC4=C3[C@@H](c3ccc(N(C)C)cc3)C[C@@]21C]

Its synthesis is described, inter alia, in patent EP 0 422 100 and in patent application EP 1 602 662.

Ulipristal acetate is a synthetic selective progesterone receptor modulator (SPRM). By virtue of its action on the progesterone receptor, ulipristal acetate is capable of exerting a contraceptive action by inhibiting or delaying ovulation. Clinical studies showed that ulipristal acetate, administered in a single dose of 30 mg, makes it possible to prevent an unwanted pregnancy when it is administered within 120 hours following unprotected or poorly protected sexual intercourse (Glasier et al, Lancet. 2010, 375(9714):555-62; Fine et al, Obstet Gynecol. 2010, 115:257-63). Ulipristal acetate has thus been authorized as an emergency contraceptive and is marketed under the trade name EllaOne® in Europe.

Other therapeutic applications of ulipristal acetate have been proposed in the prior art. Recent clinical trials showed that the chronic administration of ulipristal acetate (at 5 mg or 10 mg per day) makes it possible to significantly reduce the symptoms associated with uterine fibromas and provides a therapeutic benefit which is greater than that of the reference treatment, namely leuprolide acetate (Donnez et al., N Engl J Med. 2012; 366(5):421-32). On the basis of these clinical trials, the European Medicines Agency (EMEA) authorized, in February 2012, the proprietary drug Esmya® (5 mg of ulipristal acetate) for the pre-operative treatment of symptoms associated with uterine fibromas.

The pharmaceutical compositions currently marketed comprise ulipristal acetate in a micronized form.

The proprietary drug Esmya® is provided in the form of a non-film-coated tablet comprising 5 mg of micronized ulipristal acetate combined with the following excipients: microcrystalline cellulose, mannitol, sodium croscarmellose, talc and magnesium stearate.

EllaOne® is, for its part, provided in the form of a non-film-coated tablet comprising 30 mg of micronized ulipristal acetate and the following excipients: lactose monohydrate, povidone K30, sodium croscarmellose and magnesium stearate.

Analogous pharmaceutical compositions have also been described in international application WO 2010/066749.

The development of novel galenic forms suitable for the administration of selective progesterone receptor modulators such as ulipristal acetate remains a major challenge for their therapeutic and contraceptive uses.

In this regard, there is, at the current time, a need for novel pharmaceutical formulations containing a selective progesterone receptor modulator, such as ulipristal acetate, and having suitable release properties and a suitable bioavailability.

### SUMMARY OF THE INVENTION

A subject of the present invention is a co-micronization product comprising:
an active ingredient selected from the group consisting of selective progesterone receptor modulators, metabolites thereof and mixtures thereof, and
a polymeric excipient selected from polymers based on N-vinyl-2-pyrrolidone and mixtures thereof.

In a preferred embodiment, the active ingredient is selected from the group consisting of 17α-acetoxy-11β-(4-N-methylaminophenyl)-19-norpregna-4,9-diene-3,20-dione, 17α-acetoxy-11β-(4-aminophenyl)-19-norpregna-4,9-diene-3,20-dione, ulipristal acetate and mixtures thereof.

In certain embodiments, the co-micronization product according to the invention has one or more of the following characteristics:
the “active ingredient/polymeric excipient” weight ratio is included in a range from 0.1 to 10, preferably from 0.5 to 4,
the active ingredient is ulipristal acetate and the polymeric excipient is selected from the group consisting of a crosslinked polyvinylpyrrolidone, a non-crosslinked polyvinylpyrrolidone and mixtures thereof,
the co-micronization product may also comprise a solid surfactant, preferably sodium dodecyl sulfate,
a d50 of less than 20 μm, preferably less than 15 μm, and/or a d90 of less than 50 μm, preferably less than 40 μm.

A subject of the present invention is also a method for preparing a co-micronization product as previously defined, comprising the steps consisting in:
a) providing an active ingredient as defined above;
b) mixing the active ingredient of step a) with a polymeric excipient as defined above and, optionally, with a solid surfactant, preferably sodium dodecyl sulfate; and
c) co-micronizing the mixture obtained in step b).

An additional subject according to the invention is a pharmaceutical composition comprising the co-micronization product and a pharmaceutically acceptable excipient. The pharmaceutically acceptable excipient can be selected from the group consisting of a diluent, a binder, a flow agent, a lubricant, a disintegrant and mixtures thereof.

In certain embodiments, the pharmaceutical composition according to the invention comprises:
0.5% to 80% of co-micronization product,
15% to 95% of diluent, and
0% to 5% of lubricant,
   the percentages being expressed by weight relative to the total weight of the composition.

The pharmaceutical composition according to the invention may comprise from 1 mg to 100 mg, preferably from 1 mg to 40 mg, of active ingredient per dose unit. It may be suitable for oral administration and may be in the form of a powder, a granule, a film-coated or uncoated tablet, or a capsule.

A subject of the present invention is also a co-micronization product or a pharmaceutical composition, as previously defined, for use as a contraceptive, for example, as a regular contraceptive or as an emergency contraceptive.

Finally, a subject of the invention is also a co-micronization product or a pharmaceutical composition, as previously defined, for use in the treatment or prevention of a gynaecological disorder, preferably affecting the uterus.

### FIGURES

FIG. 1 shows the in vitro dissolution curves for various comicronizates (see Example 1 hereinafter): UPA/povidone 7/3 (open square), UPA/crospovidone (solid square), UPA/Kollicoat® IR 7/3 (cross), UPA/citric acid monohydrate 7/3 (open diamond), UPA/fumaric acid 7/3 (open circle). Control experiment: micronized UPA (alone—in the absence of excipient) (solid diamond). y-axis: percentage of UPA released (%), x-axis: time in minutes.

FIG. 2 shows the in vitro dissolution curves for various active ingredient matrices: comicronizate UPA/crospovidone/SLS 5/2/3 (open triangle), comicronizate UPA/crospovidone 7/3 (solid square), comicronizate mixture UPA/crospovidone+SLS (5/2/3) (cross). Control experiment: micronized UPA (alone—in the absence of excipient—(solid circle)). y-axis: percentage of UPA released (%), x-axis: time in minutes.

### DETAILED DESCRIPTION OF THE INVENTION

Co-Micronization Product According to the Invention and Preparation Method

At the end of lengthy research, the applicant showed that it is possible to significantly improve the in vitro dissolution profile of ulipristal acetate (hereinafter UPA) by virtue of a co-micronization technology. The applicant showed that the product resulting from the co-micronization of ulipristal acetate with a polymeric excipient of polyvinylpyrrolidone type had in vitro dissolution properties—in particular a degree of dissolution at 45 minutes—which were significantly higher than that of UPA micronized alone, in the absence of excipient. This improvement in the in vitro dissolution properties of UPA is expected to correlate with an improvement in the in vivo bioavailability. By virtue of its improved properties, the co-micronization product could make it possible to reduce the doses of UPA to be administered to the patient in order to obtain the desired therapeutic or contraceptive effect. The decrease in the dose of UPA should make it possible, inter alia, to increase the safety, in particular the innocuousness, of the final pharmaceutical compositions. Notably, the co-micronization of UPA does not systematically result in an improvement in its dissolution properties. The polymeric co-micronization excipients tested by the applicant, other than the N-vinylpyrrolidone polymers (see Example 1, Table 1 hereinafter), did not enable to improve the in vitro dissolution properties of UPA.

Moreover, unexpectedly, the co-micronization of UPA with an organic acid resulted in a clear decrease in the in vitro dissolution rate of UPA (see Example 1 hereinafter). Moreover, the product obtained by co-micronization of UPA with a polymeric excipient of PEG-PVA type exhibits an in vitro dissolution rate for UPA which is much lower than that observed for micronized UPA.

Thus, a subject of the present invention is a novel galenic form, more specifically a co-micronization product comprising:
an active ingredient selected from the group consisting of selective progesterone receptor modulators, metabolites thereof and mixtures thereof, and
an N-vinyl-2-pyrrolidone-based polymeric excipient.

The term “co-micronization product” (also hereinafter denoted comicronizate) is intended to mean the product obtained by micronization of a mixture comprising an active ingredient and at least one excipient.

The term “micronization” is intended to mean a method which makes it possible to reduce the size of the particles of a powder, for example by milling.

The reduction in the size of the particles is brought about by a decrease of at least 10% of a parameter selected from the d50, the d10 and the d90. A reduction of “at least 10%” encompasses a reduction of at least 20%, of at least 30%, and of at least 40%. The micronization can be carried out by means of commercially available devices, such as ball or air jet micronizers.

In the context of the present invention, the term “micronized product” is intended to mean a product which is in the form of a powder having a d90 of less than 50 μm. Thus, preferably, the co-micronization product according to the invention has a d90 of less than 50 μm.

The expression “a co-micronization product having a d90 of less than 50 μm” is intended to mean a co-micronization product, in the form of a powder, in which at least 90% of the particles have a size of less than 50 μm.

The expression “N-vinyl-2-pyrrolidone-based polymeric excipient”, or alternatively “N-vinyl-2-pyrrolidone-based polymer” is intended to mean a polymer comprising N-vinyl-2-pyrrolidone as monomer. Such a polymer encompasses N-vinyl-2-pyrrolidone homopolymers and N-vinyl-2-pyrrolidone copolymers. The polymeric excipient may be crosslinked or non-crosslinked.

The term “copolymer” is intended to mean a polymer comprising at least two distinct types of monomers. This may involve “random” copolymers, wherein the various types of monomers are randomly linked together, or else “block” polymers. An example of an N-vinyl-2-pyrrolidone-based copolymer is, for example, copovidone, which is a copolymer of N-vinyl-2-pyrrolidone and vinyl acetate.

In certain embodiments, the polymeric excipient is selected from the group consisting of a crosslinked polyvinylpyrrolidone (hereinafter referred to as crospovidone), a non-crosslinked polyvinylpyrrolidone (hereinafter referred to as povidone), a copovidone and mixtures thereof.

Preferably, the polymeric excipient is selected from the group consisting of a crosslinked polyvinylpyrrolidone, a non-crosslinked polyvinylpyrrolidone, a copovidone and mixtures thereof.

For the purposes of the invention, a non-crosslinked polyvinylpyrrolidone is composed of free polymer chains, not linked to one another by covalent bonds.

A crosslinked polyvinylpyrrolidone is a network consisting of polymer chains linked to one another by covalent bonds.

The crosslinked or non-crosslinked polyvinylpyrrolidones are commercially available.

By way of example, mention may be made of the crospovidone Polyplasdone® XL-10 marketed by ISP and the povidone Plasdone® K29-K32 marketed by BASF.

In certain embodiments, the polymeric excipient is selected from povidones having an average molecular weight ranging from 10³ to 10⁷ g·mol⁻¹, preferably ranging from 3×10⁴ to 9×10⁴ g·mol⁻¹. A povidone having an average molecular weight ranging from 3×10⁴ to 9×10⁴ g·mol⁻¹ encompasses an average molecular weight ranging from 30 000 to 40 000 g·mol⁻¹, from 40 000 to 50 000 g·mol⁻¹, from 50 000 to 60 000 g·mol⁻¹, from 60 000 to 70 000 g·mol⁻¹, from 70 000 to 80 000 g·mol⁻¹ and from 80 000 to 90 000 g·mol⁻¹. By way of example, a suitable polymeric excipient may be a povidone having an average molecular weight ranging from 55 000 to 65 000 g·mol⁻¹.

In other embodiments, the polymeric excipient is selected from the group consisting of crospovidones.

The term “selective progesterone receptor modulator” is intended to mean a progesterone receptor ligand which exerts an agonist activity, an antagonist activity or a mixed agonist/antagonist activity in a tissue-specific manner, preferably an agonist or a mixed agonist/antagonist activity. By virtue of their general knowledge, those skilled in the art will be able to determine, by means of routine experiments, whether a compound is an SPRM, in particular by referring to the articles by Smith and O'Malley, Endocrine Review, 25(1):45-71, and by Chabbert-Buffet et al., Human Reproduction Update, 2005, 11, 293-307.

Preferably, the SPRM compound is a steroidal derivative. Examples of steroidal SPRMs are provided in the following publications: Rao et al., Steroids, 1998, 63:523-530 and Chabbert-Buffet et al., Human Reproduction Update, 2005, 11, 293-307. In particular, Chabbert-Buffet et al. mention mifepristone, onapristone, asoprisnil, ulipristal acetate, Org 33628 and Org 31710 as being SPRMs.

The specific progesterone receptor modulators are preferably steroidal derivatives substituted in the 11β position with an aryl group.

Thus, in an advantageous embodiment, the SPRM(s) present in the comicronizate is (are) selected from the compounds of formula (I) below:

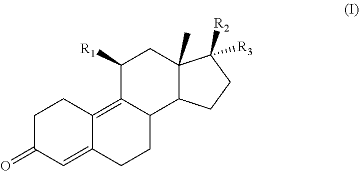

in which:
R₁ represents an aryl group optionally substituted with one or more groups independently in the ortho, para or meta position,
R₂ represents —OH, a C₁-C₅ alkoxy group or —C(O)—R₄, and
R₃ represents —OH, a C₁-C₅ alkoxy group, a C₂-C₅ alkynyl, a C₂-C₅ alkenyl or —O—C(O)—R₅,
R₄ and R₅ being selected, independently of one another, from a C₁-C₃ alkyl group and a C₁-C₅ alkoxy group,
   and also the pharmaceutically acceptable salts thereof.

A C₁-C₃ alkyl encompasses methyl, ethyl, propyl and isopropyl groups.

A C₁-C₅ alkoxy group encompasses the groups of formula —(CH₂)ₙO(CH₂)_{(y-n)}CH₃, n being an integer ranging from 0 to 4, y being an integer from 0 to 4, it being understood that (y−n) is greater than or equal to 0.

In one preferred embodiment, the SPRM is selected from the group of SPRM compounds of formula (Ia) below:

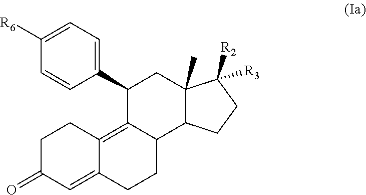

in which:
R₆ represents:
   —NR₇R₈ in which R₇ and R₈ represent, independently of one another, —H or a C₁-C₃ alkyl, R₇ and R₈ preferably being selected from H and —CH₃;
   —CH═N—O—R₉ in which R₉ represents —H or —C(O)—X—R₁₀ with R₁₀ being a C₁-C₃ alkyl and X representing 0, NH or S; or
   —C(O)R₁₁ in which R₁₁ represents a C₁-C₃ alkyl;
R₂ and R₃ being as previously defined.

Such compounds comprise, without being limited thereto, mifepristone, ulipristal acetate, asoprisnil and telapristone. In particular, mifepristone corresponds to the compound of formula (Ia) in which R₆ is —N(CH₃)₂, R₂ is OH and R₃ is —C≡C—CH₃.

In one preferred embodiment, the SPRM is selected from the group of SPRM compounds of formula (Ia) in which:
R₂ represents —OH, —OCH₃, —C(O)—CH₃ or —C(O)—CH₂—O—CH₃
R₃ represents —CH₂—O—CH₃, or —O—C(O)—CH₃
R₆ represents —NH₂, —NHCH₃, —N(CH₃)₂ or —CH═N—OR₉ in which R₉ represents H, —C(O)—S—C₂H₅ or —C(O)—NH—C₂H₅.

This group of compounds encompasses, inter alia, telapristone, asoprisnil and ulipristal acetate, and some metabolites thereof.

In certain embodiments, the SPRM is selected from the compounds of formula (Ia) in which R₆ is —CH═N—O—R₉. Such compounds encompass:
Asoprisnil (R₉ is H, R₂ is OMe and R₃ is —CH₂OMe),
J912 (R₉ is H, R₂ is H and R₃ is —CH₂OMe),
J956 (also known as Asoprisnil ecamate) (R₉ is —C(O)—NH—C₂H₅, R₂ is —OMe and R₃ is —CH₂OMe), and
J1042 (R₉ is —C(O)—S—C₂H₅, R₂ is —OCH₃ and R₃ is —CH₂OMe).

In other embodiments, the SPRM is selected from the compounds of formula (Ia) in which R₆ is —NH₂, —NHCH₃, or —N(CH₃)₂.

In one preferred embodiment, the active ingredient present in the comicronizate is selected from the group consisting of ulipristal acetate, ulipristal acetate metabolites and mixtures thereof.

UPA metabolites are described, inter alia, in Attardi et al., Journal of Steroid Biochemistry and Molecular Biology, 2004, 88: 277-288 and illustrated hereinafter:

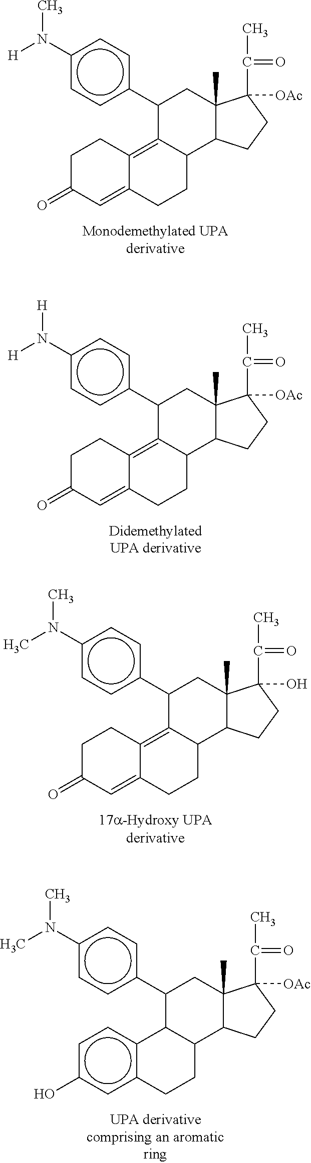

Preferably, the ulipristal acetate metabolite is selected from:
17α-acetoxy-11β-(4-N-methylaminophenyl)-19-norpregna-4,9-diene-3,20-dione (monodemethylated derivative) and
17α-acetoxy-11β-(4-aminophenyl)-19-norpregna-4,9-diene-3,20-dione (didemethylated derivative).

In a preferred embodiment of the comicronizate according to the invention, the active ingredient is selected from the group consisting of 17α-acetoxy-11β-(4-N-methylaminophenyl)-19-norpregna-4,9-diene-3,20-dione, 17α-acetoxy-11β-(4-aminophenyl)-19-norpregna-4,9-diene-3,20-dione, ulipristal acetate and mixtures thereof.

In another preferred embodiment, the co-micronization product according to the invention comprises:
an active ingredient selected from the group consisting of ulipristal acetate, 17α-acetoxy-11β-(4-N-méthylaminophenyl)-19-norpregna-4,9-diene-3,20-dione, 17α-acetoxy-11β-(4-aminophenyl)-19-norpregna-4,9-diene-3,20-dione, and mixtures thereof, and
a polymeric excipient selected from the group consisting of crosslinked polyvinylpyrrolidones, non-crosslinked polyvinylpyrrolidones and mixtures thereof.

In one additional embodiment, the co-micronization product according to the invention comprises:
an active ingredient, namely ulipristal acetate, and
a polymeric excipient selected from crosslinked polyvinylpyrrolidones and mixtures thereof.

By way of example, the co-micronization product according to the invention may comprise ulipristal acetate as active ingredient and a crospovidone as polymeric excipient.

The weight ratio between the active ingredient and the polymeric excipient in the comicronizate according to the invention may be included in a range from 0.1 to 10, preferably from 0.5 to 4. An “active ingredient/polymeric excipient” weight ratio of from 0.5 to 4 encompasses a weight ratio of from 0.5 to 1, from 1 to 1.5, from 1.5 to 2, from 2 to 2.5, from 3 to 3.5, and from 3.5 to 4. Preferably, the “active ingredient to polymeric excipient” weight ratio is included in a range from 1.5 to 4. A suitable “active ingredient/polymeric excipient” weight ratio is, for example, a weight ratio of approximately 2.3.

In certain embodiments, the comicronizate according to the invention may comprise an additional excipient. This additional excipient may make it possible to potentiate the action of the povidone or of the crospovidone on the dissolution properties of ulipristal acetate. This excipient may be selected from the surfactants commonly used in galenics and which can undergo co-micronization, typically by milling.

The term “solid surfactant” is intended to mean a surfactant which is solid at ambient temperature, i.e. typically at approximately 20° C. In certain advantageous embodiments, the surfactant has a high melting point, preferably above 50° C. and even more preferably above 100° C. The surfactant may be selected from C₈-C₂₀, preferably C₁₀-C₁₄, alkyl sulfate salts, and mixtures thereof.

In one advantageous embodiment, the surfactant is selected from the dodecyl sulphate salts, preferably the alkali metal or alkaline-earth metal salts thereof, such as a sodium, magnesium or calcium salt. A surfactant which is particularly suitable for obtaining a co-micronization product according to the invention is SDS, i.e. sodium dodecyl sulfate. Thus, in a preferred embodiment, the surfactant is sodium dodecyl sulfate.

In one particular embodiment, the co-micronization product comprises:
an active ingredient selected from the group consisting of ulipristal acetate, 17α-acetoxy-11β-(4-N-methylaminophenyl)-19-norpregna-4,9-diene-3,20-dione, 17α-acetoxy-11β-(4-aminophenyl)-19-norpregna-4,9-diene-3,20-dione, and mixtures thereof, preferably ulipristal acetate,
a polymeric excipient selected from crosslinked polyvinylpyrrolidones, non-crosslinked polyvinylpyrrolidones and mixtures thereof, and
a solid surfactant, preferably SDS.

The weight ratio between the active ingredient and the surfactant is generally included in a range from 0.1 to 10, preferably from 0.5 to 4. A suitable “active ingredient/surfactant” weight ratio is, for example, a weight ratio of approximately 1.7.

By way of example, a comicronizate according to the invention may comprise an active ingredient, a polymeric excipient and a surfactant in amounts which correspond to the following weight ratios:
an “active ingredient/surfactant” weight ratio of 1 to 2, preferably approximately 1.7, and
an “active ingredient/polymeric excipient” weight ratio of 2 to 4, preferably approximately 2.5.

The granulometry (i.e. the distribution of the size of the particles) of the co-micronization product can have an effect on the UPA solubility properties.

It is preferable for the d50 of the co-micronization product to be less than 25 μm, preferably less than 20 μm, or even less than 15 μm.

A d50 of less than 15 μm encompasses a d50 of less than 12 μm, than 11 μm, than 10 μm, than 9 μm, than 8 μm, than 7 μm, than 6 μm, than 5 μm, and than 4 μm.

It is also preferable for the d90 of the co-micronization product to be less than 50 μm, or even less than 40 μm.

A d90 of less than 40 μm encompasses a d90 of less than 38 μm, than 37 μm, than 36 μm, than 35 μm, than 34 μm, than 33 μm, than 32 μm, than 31 μm, than 30 μm, than 29 μm, than 28 μm, than 27 μm, than 26 μm, than 25 μm, than 24 μm, than 23 μm, than 22 μm, than 21 μm, than 20 μm, than 19 μm, than 18 μm, than 17 μm, than 16 μm, than 15 μm, than 14 μm, than 13 μm, than 12 μm, than 11 μm, and than 10 μm.

In certain embodiments, the co-micronization product according to the invention is characterized in that its particle size distribution has:
a d50 of less than 20 μm, preferably less than 15 μm, and/or
a d90 of less than 50 μm, preferably less than 40 μm and even more preferably less than 30 μm.

By way of example, the comicronizate according to the invention may have a d50 of less than 5 μm and/or a d90 of less than 15 μm.

The d10 of the comicronizate according to the invention is generally greater than 0.05 μm.

In the context of the present invention, “a d50 of less than X μm” means that at least 50% of the comicronizate particles have a size of less than X μm.

“A d90 of less than Y μm” means that at least 90% of the comicronizate particles have a size of less than Y μm.

Likewise, “a d10 of greater than Z μm” means that at least 90% of the comicronizate particles have a particle size of greater than Z μm.

The granulometry—i.e. the distribution of the size of the particles—of the co-micronization product, and in particular the d90, d50 and d10 parameters, can be determined by any method known to those skilled in the art. Preferably, laser diffraction will be used.

The co-micronization product has improved active ingredient in vitro dissolution properties. In certain embodiments, the co-micronization product according to the invention is characterized in that at least 55% of the active ingredient that it contains is released within 45 minutes when said co-micronization product is subjected to an in vitro dissolution test, preferably according to the European Pharmacopoeia §2.9.3. The in vitro dissolution test can be carried out using any commercially available device. Example 1 hereinafter presents implementing conditions for determining the in vitro dissolution rate of a comicronizate according to the invention. Briefly, an amount of comicronizate representing 30 mg of active ingredient is placed in a gelatin capsule. This capsule is then placed in 900 ml of a medium buffered at gastric pH, comprising 0.1% of SDS, at 37±0.5° C., and subjected to stirring at 50 revolutions per minute (rpm) (speed of rotation of the paddles of the dissolution device). The dissolution of the active ingredient in the medium can be monitored by spectrophotometry, for example at the maximum wavelength of absorbance. For the purposes of the invention, a gastric pH is typically a pH of 1 to 3.

The expression “at least 55% of the active ingredient released within 45 minutes” encompasses at least 55%, at least 60%, at least 65%, at least 70%, at least 75%, and at least 80% of the active ingredient is released within 45 minutes.

Preferably, at least 60% of the active ingredient is released within 45 minutes.

The comicronizate product can also be characterized in that at least 45% of the active ingredient that it contains is released within 30 minutes when it is subjected to an in vitro dissolution test as previously described.

A subject of the present invention is also a method for preparing the comicronizate described above comprising the steps consisting in:
a) providing an active ingredient selected from the group consisting of ulipristal acetate, a ulipristal acetate metabolite and mixtures thereof,
b) mixing the active ingredient of step a) with a polymeric excipient, and
c) micronizing the mixture obtained in step b),
   it being understood that the active ingredient and the polymeric excipient are as previously defined.

The active ingredient provided in step a) may be in micronized or non-micronized form. Moreover, the active ingredient may be amorphous or crystalline. Preferably, the active ingredient provided in step a) is in a crystalline form.

In certain embodiments, step b) may, in addition, comprise mixing the active ingredient with an additional pharmaceutical excipient, preferably a surfactant. This mixing may be carried out before, simultaneously with or after the mixing of the active ingredient with the polymeric excipient. The additional pharmaceutical excipient may be provided in micronized or non-micronized form.

The micronization step c) may be carried out using a commercially available micronization system. It may in particular be an air jet micronizer or a ball micronizer. Those skilled in the art, by virtue of their general knowledge and the performing of routine experiments, will be able to determine the conditions for carrying out step c) in order to obtain a co-micronization product having the desired particle size distribution. By way of example, when step c) is carried out using an air jet micronizer, those skilled in the art will be able to vary the powder feed flow and the pressure of the air jets in order to modulate the particle size distribution of the final comicronizate.

Pharmaceutical Composition According to the Invention

The co-micronization product is intended mainly for therapeutic or contraceptive use. For this purpose, it can be administered directly or inserted into an administration device such as a vaginal ring, a patch, an intra-uterine device or an implant.

Generally, the co-micronization product according to the invention is integrated into a pharmaceutical composition so as to facilitate its administration. Thus, an additional subject of the present invention is a pharmaceutical composition comprising a co-micronization product as previously defined and at least one pharmaceutically acceptable excipient.

Those skilled in the art will be able to choose the excipient(s) to be combined with the co-micronization product according to the final form of the pharmaceutical composition, the desired route of administration and the desired active ingredient release profile. For this purpose, those skilled in the art will be able to refer to the following reference works: Remington: The Science and Practice of Pharmacy (Lippincott Williams & Wilkins; Twenty first Edition, 2005), and Handbook of Pharmaceuticals Excipients, American Pharmaceutical Association (Pharmaceutical Press; 6th revised edition, 2009).

The pharmaceutical composition and the comicronizate according to the invention may be administered by any route, in particular the oral, buccal, nasal, sublingual, vaginal, intra-uterine, rectal or transdermal route or by the parenteral route, for example by intravenous injection. The preferred routes of administration are the buccal, oral, intra-uterine and vaginal routes.

The pharmaceutical composition according to the invention may be in any form, for example in the form of a tablet, a powder, a capsule, a pill, a suppository, a vaginal suppository, a suspension, an aqueous, alcoholic or oily solution, a syrup, a gel, an ointment, an emulsion, a lyophilizate or an orodispersible film. The route of administration and the galenic form of the pharmaceutical composition may depend on the desired therapeutic or contraceptive effect.

In certain embodiments, the pharmaceutical composition according to the invention may be integrated into a device enabling prolonged administration of the active ingredient. The pharmaceutical composition may in particular be integrated into a vaginal ring, into an intra-uterine device, into a patch, for example a transdermal or mucoadhesive patch, or into an implant, for example an implant of contraceptive type. For examples of vaginal rings suitable for implementing the invention, reference may be made to application WO 2006/10097.

In additional embodiments, the pharmaceutical composition according to the invention is in solid form. Preferably, the pharmaceutical composition according to the invention is solid and is intended for oral administration.

In certain embodiments, the pharmaceutical composition according to the invention is characterized in that the pharmaceutically acceptable excipient is selected from the group consisting of a diluent, a binder, a flow agent, a lubricant, a disintegrant and mixtures thereof.

For the purposes of the present invention, a diluent may be one or more compounds capable of densifying the active ingredient so as to obtain the desired mass. The diluents encompass inorganic phosphates, monosaccharides and polyols such as xylitol, sorbitol, lactose, galactose, xylose or mannitol, disaccharides such as sucrose, oligosaccharides, polysaccharides such as cellulose and its derivatives, starches, and mixtures thereof. The diluent may be in anhydrous or hydrated form.

By way of example, a suitable diluent according to the invention may be selected from microcrystalline cellulose, mannitol, lactose and mixtures thereof.

The binder may be one or more compounds capable of improving the aggregation of the active ingredient with the diluent. By way of example of binders, mention may be made of hydroxypropylcellulose, hydroxypropylmethylcellulose, povidone (polyvinylpyrrolidone), copolymers of N-vinyl-2-pyrrolidone and of vinyl acetate (copovidone), and mixtures thereof.

The lubricant may be one or more compounds capable of preventing the problems associated with the preparation of dry galenic forms, such as the sticking and/or gripping problems which occur in machines during compression or filling. The preferred lubricants are fatty acids or fatty acid derivatives, such as calcium stearate, glyceryl monostearate, glyceryl palmitostearate, magnesium stearate, zinc stearate, or stearic acid, polyalkylene glycols, in particular polyethylene glycol, sodium benzoate or talc. The lubricants that are preferred according to the invention are the stearate salts and mixtures thereof. A suitable lubricant is, for example, magnesium stearate.

The flow agent optionally used according to the invention may be selected from compounds which contain silicon, for example talc, anhydrous colloidal silica or precipitated silica.

The disintegrant can be used to improve the release of the active ingredient. It may be selected, for example, from crosslinked polyvinylpyrrolidone (crospovidone), crosslinked carboxymethylcellulose (such as sodium croscarmellose) or non-crosslinked carboxymethylcellulose, starches, and mixtures thereof. The disintegrant is preferably selected from the group consisting of a sodium croscarmellose, a crospovidone and mixtures thereof.

In certain embodiments, the composition according to the invention comprises:
0.5% to 80% of the co-micronization product as previously defined,
15% to 95% of diluent, preferably selected from mannitol, lactose, microcrystalline cellulose and mixtures thereof, and
0% to 5% of lubricant, preferably a stearate, such as magnesium stearate,
   the percentages being expressed by weight relative to the total weight of the composition.

The composition according to the invention may in addition be characterized in that it comprises from 0% to 20% by weight of a binder, from 0% to 10% of a disintegrant and from 0% to 5% by weight of a flow agent.

It should be noted that the weight percentage of disintegrant of the pharmaceutical composition according to the invention does not take into account the crospovidone optionally contained in the comicronizate. Likewise, the weight percentage of binder of the pharmaceutical composition according to the invention does not comprise the povidone optionally contained in the comicronizate.

In other embodiments, the pharmaceutical composition according to the invention comprises from:
1% to 60% by weight of co-micronization product as previously described,
30% to 95% by weight of diluent,
0% to 10% by weight of disintegrant,
0% to 10% by weight of a binder,
0% to 5% by weight of a flow agent, and
0.1% to 2% of a lubricant,
   the percentages being expressed by weight relative to the total weight of the composition.

The pharmaceutical composition according to the invention may also comprise one or more excipients in addition to the abovementioned excipients. The additional excipient(s) may be selected from the group consisting of surfactants such as SDS, coating agents, such as coating agents based on polyvinyl alcohol or on hydroxypropylmethylcellulose, pigments such as aluminium oxide or iron oxide, flavourings, wetting agents, waxes, dispersants, stabilizers and preservatives.

The pharmaceutical composition according to the invention may be prepared according to any one of the methods commonly used in galenics. These methods typically comprise mixing the co-micronization product according to the invention with one or more excipients, then shaping the mixture obtained. By way of example, when it is in the form of a tablet, the pharmaceutical composition according to the invention can be prepared by direct compression or by compression after dry or wet granulation.

The co-micronization product present in the pharmaceutical composition according to the invention may have any one of the characteristics described in the present description. In particular, the co-micronization product of the pharmaceutical composition has one or more (1, 2, 3, 4, 5, 6, 7 or 8) of the following characteristics:
i. the active ingredient is UPA,
ii. the polymeric excipient is selected from the group consisting of povidones, crospovidones and mixtures thereof,
iii. the “active ingredient/polymeric excipient weight ratio” is included in a range from 0.5 to 4,
iv. it also comprises a surfactant, preferably SDS,
v. when the surfactant is present, the “active ingredient/surfactant weight ratio” is from 0.5 to 4,
vi. the d50 of the co-micronization product is less than 20 μm, preferably less than 15 μm,
vii. the d90 of the co-micronization product is less than 50 μm, preferably less than 40 μm, and
viii. at least 55% of the active ingredient that the co-micronization product contains is released within 45 minutes when said co-micronization product is subjected to an in vitro dissolution test, preferably under the following conditions:
   device: paddle dissolution device,
   sample: hard gelatin capsule containing an amount of comicronizate corresponding to 30 mg of active ingredient,
   dissolution medium: 900 ml of an aqueous solution buffered at gastric pH comprising 0.1% of SDS,
   temperature: 37±0.5° C., and
   paddle rotation speed: 50 revolutions per minute (rpm).

As previously mentioned, the composition according to the invention may be in the form of a powder, a granule, a film-coated or uncoated tablet, or a capsule, and is preferably intended for oral administration. In certain embodiments, the pharmaceutical composition according to the invention is in the form of an uncoated tablet intended for oral administration.

The composition according to the invention may be a controlled-, immediate-, sustained- or delayed-release pharmaceutical composition. Preferably, the composition according to the invention is an immediate-release composition.

The term “immediate-release composition” is intended to mean a pharmaceutical composition characterized in that at least 75% of the active ingredient initially contained in a dose unit of the pharmaceutical composition is released within 45 minutes when said dose unit is subjected to an in vitro dissolution test, for example according to the European Pharmacopoeia §2.9.3, and preferably under the following conditions:
paddle dissolution device,
dissolution medium: aqueous solution buffered at gastric pH containing 0.1% of SDS,
temperature: 37±0.5° C., and
rotation speed: 50 rpm.

The volume of the dissolution medium depends on the amount of active ingredient contained in the dose unit. For a dose unit comprising 5 mg of active ingredient, 500 ml of dissolution medium are used. For a dose unit comprising 30 mg of active ingredient, 900 ml of dissolution medium are used.

Generally, the pharmaceutical composition comprises from 1 mg to 100 mg of active ingredient per dose unit, preferably from 1 mg to 40 mg, or even from 2 mg to 30 mg, of active ingredient per dose unit. The dose of active ingredient depends on the therapeutic or contraceptive effect and on the administration scheme that are desired. For example, for certain applications, the amount of UPA per dose unit may be included in a range from 1 mg to 5 mg.

In emergency contraception, the active ingredient may be present in an amount of from 20 mg to 40 mg per dose unit.

In regular contraception, the active ingredient may be present in an amount of from 2 mg to 5 mg per dose unit.

For therapeutic uses such as the treatment of uterine fibromas, the active ingredient may be present in an amount of from 3 mg to 15 mg per dose unit.

The dose of active ingredient and the administration scheme may also depend on the personal parameters of the patient, in particular the weight, age, sex, general health condition and diet, on the pathological conditions from which the patient is suffering, etc.

Finally, the pharmaceutical composition according to the invention may comprise an additional active ingredient. This additional active agent may exert an action different from that of UPA or its metabolites. It may also reinforce the therapeutic effect of UPA or its metabolites.

Therapeutic or Contraceptive Uses of the Comicronizate and of the Pharmaceutical Composition According to the Present Invention

In an additional aspect, a subject of the present invention is also a co-micronization product or a pharmaceutical composition as previously described, for use as a medicament. The co-micronization product or the composition according to the invention is particularly suitable for use as a regular contraceptive or an emergency contraceptive. It can also be used for the treatment or prevention of hormonal, gynaecological or endocrine disorders, such as Cushing's disease. The composition or the co-micronization product according to the invention can be used, in particular, in the treatment or prevention of a gynaecological disorder, preferably affecting the uterus, including benign gynaecological disorders. The gynaecological disorders encompass, without being limited thereto, uterine fibromas and symptoms thereof, adenomyosis, endometriosis, pain associated with endometrium dislocation, and excessive uterine bleeding.

An additional subject of the invention is the use of the co-micronization product according to the invention for preparing a contraceptive or for preparing a medicament intended for the treatment or prevention of any one of the abovementioned pathological conditions.

A subject of the invention is also a method of contraception comprising the administration, to a patient, of a contraceptive dose of the co-micronization product or of the pharmaceutical composition according to the invention.

The term “method of contraception” is intended to mean a method which makes it possible to prevent the occurrence of a pregnancy in a patient of child-bearing age.

In the case in point, it may be a method of emergency contraception. In this case, a single dose is preferably administered to the patient within an appropriate time period after unprotected or poorly protected sexual intercourse, generally within 120 h following unprotected or poorly protected sexual intercourse. The method of contraception may also be a method of regular contraception, in which the composition or the co-micronization product are administered chronically and cyclically to the patient or continuously using a device such as an implant or a vaginal ring. By way of alternative, the method of contraception may be a method of “on demand” contraception as described in international application WO 2010/119029. Finally, a subject of the invention is also a method for treating a disease or a disorder in a patient, comprising the administration of a therapeutically effective dose of the co-micronization product or of the pharmaceutical composition according to the invention to the patient, who is preferably female. The therapeutic method according to the invention preferably relates to any one of the abovementioned diseases or disorders. It goes without saying that, for the implementation of the methods and uses described above, the co-micronization product and the pharmaceutical composition according to the invention may comprise one or more of the characteristics explained in detail in the present description.

The objective of the examples hereinafter is to illustrate the invention more fully without, however, limiting the scope thereof.

### EXAMPLES

### Example 1

### Preparation of Comicronizates and Screening of Excipients

1. Materials and Methods

Preparation of Comicronizates

The ulipristal acetate co-micronization products (hereinafter “comicronizates”) were prepared according to the following method: The ulipristal acetate and the co-micronization excipient to be tested were mixed in the desired weight ratio in a mortar and triturated until a homogeneous mixture was obtained. The mixture obtained was micronized using a ball mill-homogenizer so as to obtain the desired particle size distribution.

In Vitro Dissolution of the UPA Comicronizates

For each comicronizate obtained, gelatin capsules containing an amount of comicronizate corresponding to 30 mg of UPA per capsule were prepared. The studies of the in vitro dissolution of UPA as comicronizate were carried out using these capsules according to the method described in the European Pharmacopoeia in §2.9.3, using a paddle dissolution device.

For each comicronizate, a capsule containing said comicronizate was placed in a bowl of the dissolution device containing 900 ml of a dissolution medium. The dissolution medium is an aqueous solution buffered at gastric pH and comprising 0.1% of SDS. The conditions for carrying out the in vitro dissolutions are the following:
Paddle rotation speed: 50 revolutions per minute (rpm)
Temperature: 37° C.±0.5° C.

The dissolution of the UPA was monitored by spectrophotometry.

By way of control experiment, a gelatin capsule containing 30 mg of ulipristal acetate micronized alone (i.e. UPA micronized in the absence of any co-micronization excipient) was used. For each comicronizate, the dissolution experiment was reproduced 3 times.

2. Results

Experiment Series 1: Co-Micronization Excipient Screening

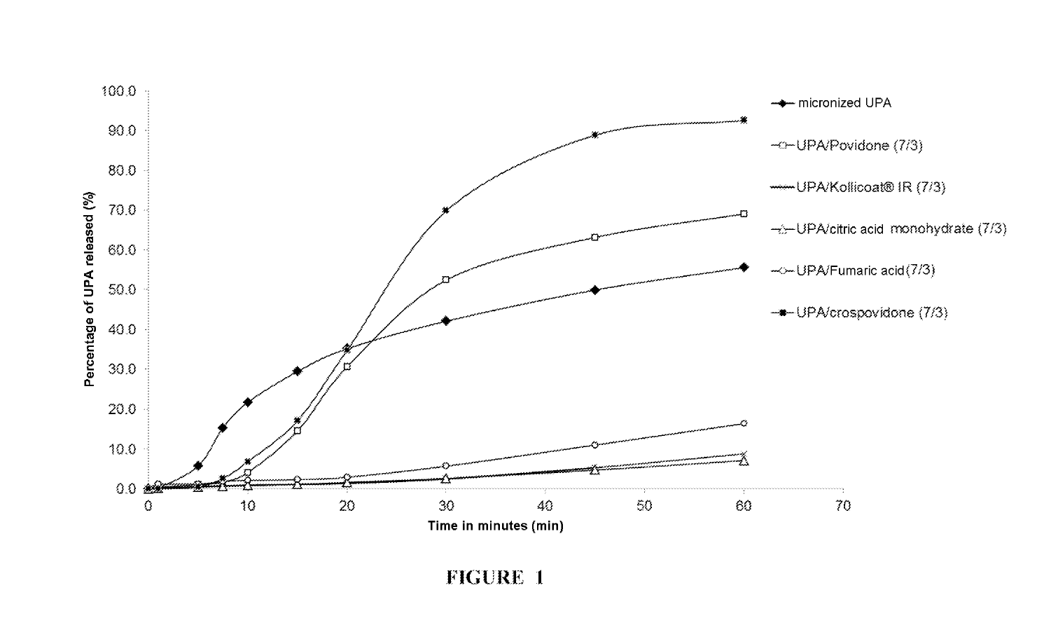

Table 1 below and FIG. 1 show the dissolution results obtained for each comicronizate prepared. The dissolution percentages are expressed relative to the initial amount of UPA contained in each capsule.

TABLE 1
Results of the in vitro dissolution assays for the UPA/excipient comicronizates prepared.
UPA/excipient weight ratio 7/3. Control experiment: UPA micronized alone.

| | Percentages of UPA released, expressed relative to the initial amount of UPA contained per gel capsule | | | | | |
|---|---|---|---|---|---|---|
| | (Mean values over 3 experiments) | | | | | |
| Time | Micronized | Povidone | Crospovidone | Kollicoat ® | Citric acid | Fumaric |
| (min) | UPA | (Plasdone ® K29-32) | (Polyplasdone ® XL10) | IR | monohydrate | acid |
| 0 | 0.00 | 0.00 | 0.00 | 0.00 | 0.00 | 0.00 |
| 1 | 0.00 | 0.40 | 0.00 | 0.20 | 0.10 | 1.10 |
| 5 | 5.50 | 1.00 | 0.50 | 0.50 | 0.50 | 1.20 |
| 7.5 | 15.00 | 1.70 | 2.60 | 0.70 | 0.60 | 1.80 |
| 10 | 22.30 | 4.10 | 6.80 | 0.90 | 0.80 | 2.10 |
| 15 | 30.00 | 14.50 | 17.10 | 1.10 | 1.10 | 2.30 |
| 20 | 34.30 | 30.60 | 34.80 | 1.40 | 1.60 | 2.90 |
| 30 | 39.50 | 52.50 | 69.90 | 2.50 | 2.60 | 5.70 |
| 45 | 47.50 | 63.20 | 88.90 | 5.30 | 4.70 | 11.00 |
| 60 | 53.80 | 69.10 | 92.60 | 8.80 | 7.10 | 16.40 |

It is specified that Kollicoat IR® is a polyethylene glycol/polyvinyl alcohol grafted copolymer.

These results show that the co-micronization of ulipristal acetate with a crospovidone or a povidone makes it possible to very significantly improve the final amount (at t=60 min) of UPA released and therefore the overall dissolution rate. Notably, the percentage of ulipristal acetate released into the medium at t=30 min is approximately 52% for a gel capsule comprising the UPA/povidone comicronizate and approximately 69% for a gel capsule comprising the UPA/crospovidone comicronizate, while it is only approximately 39% for a capsule containing micronized ulipristal acetate.

The comicronizate of UPA and Kollicoat IR® has a UPA release rate which is much lower than that observed for the micronized UPA since, after 60 min, less than 10% of the UPA initially contained in the comicronizates has been released. Since the solubility of ulipristal acetate is pH-dependent (see Table 2 below), it was expected that the co-micronization of ulipristal acetate with an acidic excipient—such as citric acid or fumaric acid—would make it possible to improve the dissolution of ulipristal acetate by decreasing the pH in the close surroundings of the dosage form and therefore by locally increasing its solubility.

TABLE 2
UPA solubility as a function of pH

| pH | UPA solubility (g/l) |
|---|---|
| 1.2 | 22.7 |
| 4.5 | 0.039 |
| 6.8 | 0.005 |

Surprisingly, the co-micronization of UPA with an organic acid leads to a clear decrease in the dissolution rate and the final degree of dissolution (at t=60 min) of UPA.

### CONCLUSION

The co-micronization in the presence of an N-vinylpyrrolidone-based polymer such as a povidone or a crospovidone made it possible to improve the in vitro UPA dissolution profile compared with UPA in micronized form. On the other hand, contrary to what might have been expected, the other co-micronization excipients tested in this example had a clearly negative impact on UPA release.

Experiment Series 2: Effects of the Addition of a Surfactant

The following comicronizates were prepared:
UPA/crospovidone 7/3 comicronizate and
UPA/crospovidone/SDS 5/2/3 comicronizate.

An active ingredient matrix was also prepared by mixing a UPA/crospovidone 5/2 comicronizate with SDS in order to obtain a 5/2/3 UPA/crospovidone/SDS mixture.

This matrix is hereinafter referred to as UPA/crospovidone/external SDS.

A micronized UPA powder was used as control experiment.

These various active ingredient matrices were subjected to an in vitro dissolution test as described above in the “Materials and Methods” section.

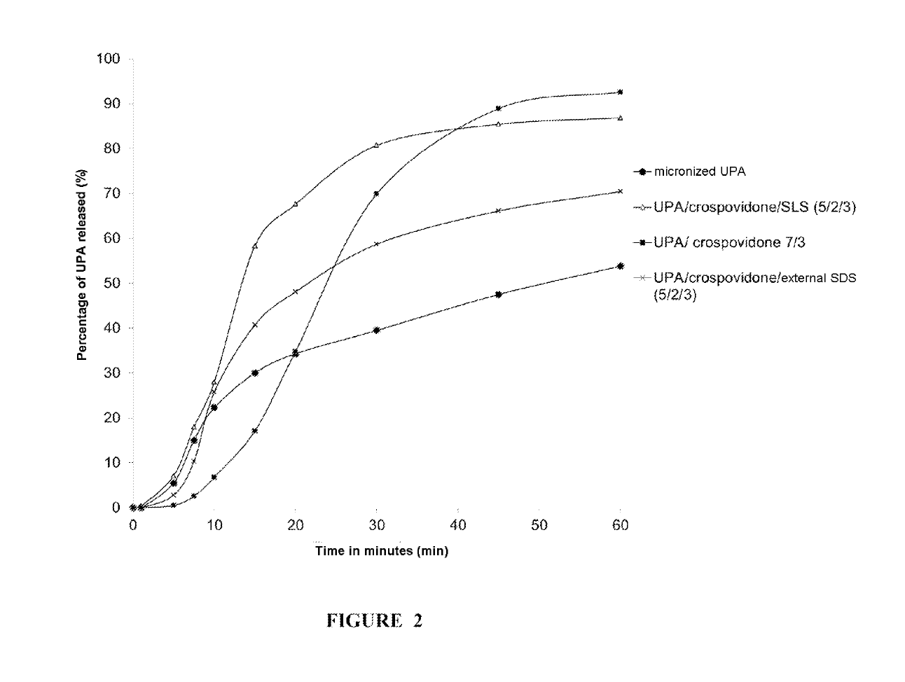

The in vitro dissolutions obtained are illustrated in FIG. 2 and presented in Table 3 hereinafter.

TABLE 3
In vitro dissolution results

| | Percentages of UPA released, expressed relative to | | | |
|---|---|---|---|---|
| | the initial amount of UPA contained per gel capsule | | | |
| | (Mean values over 3 experiments) | | | |
| | | | | UPA/cros- |
| | | | | povidone/ |
| | | | UPA/cros- | external |
| | Micron- | UPA/cros- | povidone/ | SDS (5/2/3) |
| Time | ized UPA | povidone 7/3 | SDS (5/2/3) | (invention and |
| (min) | (comparative) | (invention) | (invention) | comparative) |
| 0 | 0.00 | 0.00 | 0.00 | 0.00 |
| 1 | 0.00 | 0.00 | 0.40 | 0.00 |
| 5 | 5.50 | 0.50 | 7.10 | 2.80 |
| 7.5 | 15.00 | 2.60 | 18.00 | 10.30 |
| 10 | 22.30 | 6.80 | 28.00 | 25.80 |
| 15 | 30.00 | 17.10 | 58.30 | 40.70 |
| 20 | 34.30 | 34.80 | 67.60 | 48.10 |
| 30 | 39.50 | 69.90 | 80.70 | 58.70 |
| 45 | 47.50 | 88.90 | 85.40 | 66.10 |
| 60 | 53.80 | 92.60 | 86.80 | 70.40 |

An improvement in the in vitro dissolution is observed for all the comicronizates tested. The improvement is less marked for the UPA/Crospovidone/external SDS matrix, thereby illustrating the specific effect of the co-micronization (compared with physical mixing).

### Example 2

### Pharmaceutical Compositions Integrating the Comicronizate According to the Invention

Tables 4 and 5 hereinafter present examples of a pharmaceutical composition according to the invention. These pharmaceutical compositions can be prepared by direct compression of a mixture comprising the comicronizate and the various excipients.

TABLE 4
Example of a composition according to
the invention comprising 5 mg of UPA

| | | % by | |
|---|---|---|---|
| Ingredients | Function | weight | mg/tablet |
| Comicronizate UPA/ | Active ingredient | 4.7 | 7.1 |
| crospovidone 7/3 | matrix | | (i.e. 5 mg of UPA) |
| Microcrystalline | Diluent | 60.8 | 91.2 |
| cellulose | | | |
| Mannitol | Diluent | 29.0 | 43.5 |
| Crospovidone | Disintegrant | 4.9 | 7.4 |
| Magnesium stearate | Lubricant | 0.5 | 0.8 |

| | |
|---|---|
| Total | 150 |

This composition can be used, for example, for the treatment of uterine fibromas.

TABLE 5
Example of a composition according to
the invention comprising 30 mg of UPA

| | | % by | |
|---|---|---|---|
| Ingredients | Function | weight | mg/tablet |
| Comicronizate UPA/ | Active ingredient | 28.4 | 42.6 |
| crospovidone 7/3 | matrix | | (i.e. 30 mg of UPA) |
| Microcrystalline | Diluent | 37.1 | 55.7 |
| cellulose | | | |
| Mannitol | Diluent | 29.0 | 43.5 |
| Crospovidone | Disintegrant | 4.9 | 7.4 |
| Magnesium stearate | Lubricant | 0.5 | 0.8 |

| | |
|---|---|
| Total | 150 |

This composition can be used, for example, in emergency contraception.

1. A co-micronization product comprising:
an active ingredient selected from the group consisting of selective progesterone receptor modulators, metabolites thereof and mixtures thereof, and
a polymeric excipient selected from the group consisting of polymers based on N-vinyl-2-pyrrolidone and mixtures thereof.

2. The co-micronization product of claim 1, wherein the active ingredient is selected from the group consisting of 17α-acetoxy-11β-(4-N-methylaminophenyl)-19-norpregna-4,9-diene-3,20-dione, 17α-acetoxy-11β-(4-aminophenyl)-19-norpregna-4,9-diene-3,20-dione, ulipristal acetate and mixtures thereof.

3. The co-micronization product of claim 1, wherein the “active ingredient/polymeric excipient” weight ratio ranges from 0.1 to 10.

4. The co-micronization product of claim 1, wherein:
the active ingredient is ulipristal acetate and
the polymeric excipient is selected from the group consisting of a non-crosslinked polyvinylpyrrolidone, a crosslinked polyvinylpyrrolidone and mixtures thereof.

5. The co-micronization product of claim 1, which further comprises a solid surfactant.

6. The co-micronization product of claim 1 having:
a d50 of less than 20 μm, and/or
a d90 of less than 50 μm.

7. A method for preparing the co-micronization product as defined in claim 1, comprising the steps of:
a) providing an active ingredient as defined in claims 1;
b) mixing the active ingredient of step a) with a polymeric excipient selected from the group consisting of polymers based on N-vinyl-2-pyrrolidone and mixtures thereof and, optionally with a solid surfactant; and
c) co-micronizing the mixture obtained in step b).

8. A pharmaceutical composition comprising the co-micronization product as defined in claim 1 and a pharmaceutically acceptable excipient.

9. The pharmaceutical composition of claim 8, wherein the pharmaceutically acceptable excipient is selected from the group consisting of a diluent, a binder, a flow agent, a lubricant, a disintegrant and mixtures thereof.

10. The pharmaceutical composition of claim 8, comprising:
from 0.5% to 80% of co-micronization product,
from 15% to 95% of diluent, and
from 0% to 5% of lubricant,
the percentages being expressed by weight relative to the total weight of the composition.

11. The pharmaceutical composition of claim 8, which comprises from 1 mg to 100 mg of active ingredient per dose unit.

12. The pharmaceutical composition of claim 8, which is suitable for oral administration.

13. The pharmaceutical composition of claim 8, which is in the form of a powder, a granule, a film-coated or uncoated tablet, or a capsule.

14. A method for providing contraception to a subject, comprising administering to said subject the co-micronization product of claim 1 or a pharmaceutical composition comprising said co-micronization product.

15. A method for treating or preventing a gynaecological disorder in a subject, comprising administering to said subject the co-micronization product of claim 1 or a pharmaceutical composition comprising said co-micronization product.
